(19) United States
(12) Patent Application Publication
(10) Pub. No.: US 2026/0081013 A1
(43) Pub. Date: Mar. 19, 2026

(54) METHOD OF COMMUNICATING AND PROVIDING ROBUST CRITICAL FORMULATION OF HEALTH-RELATED CLINICAL DATA AND GUIDANCE OF A PATIENT USING CONVERSATIONAL AND ACTIONABLE ARTIFICIAL INTELLIGENCE (AI) AND NATURAL LANGUAGE PROCESSING (NLP)

(71) Applicants: Kris Narayan, San Ramon, CA (US); Kadeer Halimi, Fremont, CA (US); Vishwas Kulkami, San Ramon, CA (US); Andris Vaskis, Eastpm, PA (US); Anuj Jauhari, Sonoma, CA (US)

(72) Inventors: Kris Narayan, San Ramon, CA (US); Kadeer Halimi, Fremont, CA (US); Vishwas Kulkami, San Ramon, CA (US); Andris Vaskis, Eastpm, PA (US); Anuj Jauhari, Sonoma, CA (US)

(73) Assignee: MediKarma Inc., Danville, CA (US)

(21) Appl. No.: 18/199,958

(22) Filed: May 21, 2023

Related U.S. Application Data
(60) Provisional application No. 63/372,207, filed on Feb. 24, 2022.

(51) Int. Cl. G16H 40/67; G16H 10/20; G16H 10/60; G16H 50/30; G16H 50/70

(52) U.S. Cl. CPC G16H 40/67; G16H 10/20; G16H 10/60; G16H 50/30; G16H 50/70

(57) ABSTRACT

Embodiments of the present disclosure may include a method of mapping patient data and representing data from electronic health records of an individual through a pictorial representation of their human body including receiving, over at least one communication network from each of a plurality of user computing devices operated by each of a plurality of users, electronic health records (EHR) respectively representing information from the plurality of user's electronic medical records, procured from the user's health care provider or the user's electronic health records (EHR) partners. Embodiments may also include providing, a decentralized and distributed patient facing method of communicating and providing robust critical formulation of health-related clinical data of a patient using artificial intelligence (AI) and natural language processing (NLP) to engage a patient that encourages a change in a patient's behavior to improve the patient's overall health.

### CROSS-REFERENCE TO RELATED APPLICATIONS

CLAIM OF PRIORITY. This application claims the priority of U.S. Provisional Application No. 60/372,207 filed Feb. 7, 2022 and U.S. patent application Ser. No. 18/165,852 which are incorporated herein by reference.

### BACKGROUND

Lifestyle factors contribute to the development of many chronic diseases, and their associated morbidity and mortality. Many of these chronic health conditions may be prevented, and an individual's span and quality of life improved through changes in lifestyle, such as consuming a healthy diet, exercising regularly and eliminating the abuse of tobacco and alcohol.

In particular, cigarette smoking is the single most preventable cause of premature death in the United States. More than 430,000 Americans die each year from smoking-related illness, translating to one in every five deaths. An additional 110,000 people die of causes related to alcohol abuse.

Obesity is another major cause of morbidity and mortality in the United States. More than half of all adults in the United States are considered overweight or obese. Far from being a purely cosmetic disorder, obesity substantially increases morbidity and impairs the quality of life in affected individuals. It is also a risk factor for chronic diseases including hypertension, coronary heart disease, Type II diabetes, gallbladder disease, osteoarthritis and cancers of the breast, colon, and uterus. Thus, it is important to reduce body weight among obese and overweight individuals, and to prevent further weight gain in both normal and over-weight individuals.

Physical inactivity, defined as the absence of leisure time physical activity such as recreational exercise, is another major lifestyle-related risk factor for chronic health related conditions. In the United States and other developed countries, industrial automation has caused the majority of the population to be involved in sedentary occupations. Chronic conditions related to inactivity include coronary heart disease, hypertension, Type II diabetes, depression anxiety, osteoporotic hip fractures and obesity.

Lifestyle modifications alone successfully can be used to treat many chronic health conditions, without resorting to the usage of medication. For instance, mild hypertension is often controlled through lifestyle changes such as dietary modification, weight reduction, stress control, and physical activity. Weight control in itself delays and even prevents the onset of Type II Diabetes and mild hypertension.

In addition to lifestyle factors, the risk of developing many chronic health conditions is affected by a family history of disease. Diseases such as cancer, heart disease, elevated lipids, obesity, diabetes, stroke, high blood pressure, alcoholism, mental illness, and allergies all are affected impacted by the behaviors and habits of the patients.

### BRIEF SUMMARY

Embodiments of the present disclosure may include a decentralized and distributed method of communicating and providing robust critical formulation of health-related clinical data of a patient using artificial intelligence (AI) including conversational AI and Actionable AI and natural language processing (NLP) to engage a patient, including receiving a random query from a patient on any personal health-related concern.

Embodiments may also include accessing a decentralized network for retrieving data specific to each of a plurality of users, including electronic health records (EHR) or other Heath care data bases respectively representing information from the plurality of user's electronic medical records, procured from the user's health care provider or the user's electronic health records (EHR) partners.

Embodiments may also include accessing a decentralized network to retrieve data from a plurality of smart wearable medical or sport devices. Embodiments may also include accessing a decentralized network to retrieve proprietary questionnaire data. In some embodiments, the questionnaire data includes a daily health check survey. In some embodiments, the artificial intelligence (AI) may be used to verify identity of patient to help retrieve and integrate date from multiple sources including HER, smart devices or remote monitoring devices.

Embodiments may also include accessing historical data of all past prior patient queries in a chat window. In some embodiments, the historical data may be aggregated to train the AI to contextualize each new query. Embodiments may also include responding to the query from the patient by communicating critical health data retrieved from the decentralized network, assessing the patient's health, and providing next steps for the patient based on the patient's health assessment.

In some embodiments, the artificial intelligence (AI) may be a human-like character with an avatar representing a health care expert named “Jill”. In some embodiments, the avatar can be any character selected by a user, including any variation of customized features, selected from any one of race, gender, color, or creed. In some embodiments, the avatar uses augmented reality technology to create an animation for the presentation of the response to the patient query.

In some embodiments, the response to the patient may include a plurality of ailments and conditions and further providing an explanation and/or prognosis of steps the patient may be recommended to take to address the plurality of ailments and conditions. In some embodiments, the artificial intelligence and natural language processing databases are web 3.0 compliant. In some embodiments, the response to the patient may include educational content or navigational guidance to help patient use the Platform and its contents.

In some embodiments, the avatar provides pre-operation or post-operation instructions to the patient upon receiving the patient query. In some embodiments, the decentralized network may include a library of frequently asked questions (FAQ) for all known health standards to date and may be trained according to standards stated in any one of the National Library of Medicine, Mayo Clinic, World Health Organization (WHO), WebMD, MedicinePlus or any other reputable authorities in medical training.

In some embodiments, the artificial intelligence and natural language processing database may be equipped to identify and perform prescription refills utilizing prescription data. In some embodiments, the prescription refill data includes which pharmacy chains and that may be available to fill the prescription. In some embodiments, the prescription refill data includes the location of the pharmacy available to fill the prescription.

In some embodiments, the avatar identifies and provides preventive care through knowledge of the patient's chronic conditions and proactive/preventive tests and diagnostic tests and/or required vaccinations. In some embodiments, the artificial intelligence avatar interacts with a patient avatar to present a representation of all of the ailments and conditions relevant to the patient. Embodiments may also include electronic health records (EHR) respectively representing information from the plurality of user's electronic medical records, procured from the user's health care provider or the user's electronic health records (EHR) partners, generate a health score to alert the patient of patient's overall health assessment.

Embodiments of the present disclosure may also include a system including a computing device configured to access non-transitory processor-readable media having instructions that, when executed by the computing device cause the computing device to provide a decentralized and distributed method of communicating and providing robust critical formulation of health-related clinical data of a patient using artificial intelligence (AI) and natural language processing (NLP) to engage a patient, including receiving a random query from a patient on any personal health-related concern.

Embodiments may also include accessing a decentralized network for retrieving data specific to each of a plurality of users, including electronic health records (EHR) respectively representing information from the plurality of user's electronic medical records, procured from the user's health care provider or the user's electronic health records (EHR) partners.

Embodiments may also include accessing a decentralized network to retrieve data from a plurality of smart wearable medical or sport devices. Embodiments may also include accessing a decentralized network to retrieve proprietary questionnaire data. In some embodiments, the questionnaire data includes a daily health check survey.

Embodiments may also include accessing historical data of all past prior patient queries in a chat window. In some embodiments, the historical data may be aggregated to train the AI to contextualize each new query. Embodiments may also include and. Embodiments may also include responding to the query from the patient by communicating critical health data retrieved from the decentralized network, assessing the patient's health, and providing next steps for the patient based on the patient's health assessment. Additionally, the artificial intelligence (AI) may be a human-like character with an avatar representing a health care expert character named “Jill”.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an aspect of the subject matter in accordance with one embodiment.

FIG. 2 illustrates an aspect of the subject matter in accordance with one embodiment.

FIG. 3 illustrates an aspect of the subject matter in accordance with one embodiment.

FIG. 4 illustrates an aspect of the subject matter in accordance with one embodiment.

FIG. 5 illustrates an aspect of the subject matter in accordance with one embodiment.

FIG. 6 illustrates an aspect of the subject matter in accordance with one embodiment.

FIG. 7 illustrates an aspect of the subject matter in accordance with one embodiment.

FIG. 8 illustrates an aspect of the subject matter in accordance with one embodiment.

FIG. 9 illustrates an aspect of the subject matter in accordance with one embodiment.

FIG. 10 illustrates an aspect of the subject matter in accordance with one embodiment.

### DETAILED DESCRIPTION

In the following description, a source of personalized health content is understood to include a record of the content, such as an HTML file and a server, respectively, to provide non-limiting examples of health-related data for obtaining an overall health score. Personalized health content is understood to refer generally to health content that is personalized to an individual's profile and general health attributes, and not merely to a health situation of a generic individual having a given health related condition. In a system for delivering health information to, and monitoring a plurality of individuals, personalized health content is understood to refer to health content customized to each individual. The statement that an input device is in communication with some data processing means is understood to mean that the data processing means is adapted to use data specified by the input either directly or indirectly. The term server is under-stood to refer to an information-generating device capable of communicating with a plurality of clients; servers include computer servers and television delivery systems. The term individual is understood to refer to a person at risk for engaging in behavior having adverse health consequences, as well as a person suffering from a chronic condition or disease. An exemplary embodiment discussed below describes a computer-based implementation of a system and method for general personal health improvement with special emphasis on physical activity, medication, diet and nutrition. It will be clear to an individual with ordinary skill in the art that the present invention is suitable for preventive care directed to many other health conditions. Moreover, there are many well-known structures, interfaces and processes that are suitable for implementing the present invention.

Jill is a patient-facing healthcare chatbot designed to integrate and analyze various sources of health data, including electronic medical records (EMRs), health questionnaires, data from wearable devices, and smart medical equipment. The chatbot utilizes advanced natural language processing (NLP) techniques to communicate with users, providing personalized health insights, recommendations, and educational information based on their specific health conditions, medical history, and lifestyle factors. With a focus on user-friendliness, security, and privacy, Jill empowers individuals to better manage their health, particularly for those with chronic diseases such as diabetes, hypertension, obesity, stroke, and kidney disease. Jill incorporates unique conversational Artificial intelligence and Actionable Artificial intelligence compared to current ChatGPT, GPT or other LLM based solutions that primarily delve in assembling and providing data in a query format.

The MediKarma platform, which hosts Jill, is a cloud-based application that generates health scores using medical records, smart health device data, and questionnaires. The platform is designed to be comprehensive and user-friendly, providing users with dashboards and screens for a better understanding of their health and medical history. It offers a free service to users, with revenue generated from partnerships in telehealth, telemedicine, and other healthcare services.

Key aspects of Jill and the MediKarma platform include:
Data Integration: Electronic Medical Records (EMRs); direct connections to certain EMRs; Third-party vendors for other EMRs; and user consent and identity verification for medical record requests.
Health Devices and Applications: Fitbit, Apple Health, Google Fit, Samsung Health, Garmin, etc.; and Smart glucometers, scales, and other medical equipment.
Health Questionnaires and Daily Health Check Surveys.
Future Integrations: Insurance claim and payment data; and Pharmacy prescription and refill data.
AI Chatbot-Jill: Advanced Natural Language Processing (NLP); Personalized Health Insights and Recommendations; Educational Content Delivery; Navigation Assistance within the Application; Booking appointments (e.g., vaccines, provider visits); Voice and Text Interaction.
Security and Privacy: Fully HIPAA compliant; Industry-standard security practices; and Secure management of sensitive health data.
User Experience: Seamless integration within the MediKarma platform; Contextual and personalized conversation based on user's health data; Voice and text interaction options for enhanced accessibility; Assistance in navigating the platform and finding relevant information; Proactive recommendations for better health management and preventative care; and Easy access to booking appointments and finding healthcare resource.
Target Audience: Focused on individuals with chronic diseases; Focused on individuals interested in understanding their health; and Focused on individuals interested in improving their health.
Preventative Care: Proactive recommendations from Jill; Advice on the management of health conditions; and Promotion of healthier lifestyle choices.
Future Enhancements: Refinement of suggestions and comments based on user data trends; Expansion to a broader audience beyond chronic disease management; and Continuous improvement and growth of Jill's capabilities and knowledge.
Technological Infrastructure: Combination of algorithms and techniques in chatbot development; Utilization of existing frameworks and libraries as a foundation; Contextual understanding of user's health data during interactions; and Ranking of different health data sources based on trust levels.
User Onboarding and Data Retrieval: Users prompted to add medical information through multiple methods; User authentication through EMR patient portals; Request for medical records via the Care Quality Network; Requires us to complete identity verification; Connection of smart health devices and applications; and Repetition of the process to ensure comprehensive data collection.
Voice Interaction and Data Processing: Voice data or text captured at the user's device; Conversion of voice data into text at the server-side; Identification of user intent and generation of an appropriate response; and Provision of both audio and text data in the response.
Improvements: Working with internal clinical department to create new ways of providing user feedback; Integration of expert knowledge and insights into Jill's recommendations; and Development of new features and enhancements to improve user experience.
Long-term Vision and Roadmap: Expansion of Jill's capabilities to provide more personalized insights and recommendations; Integration of additional data sources for a more holistic understanding of user health, i.e., Insurance claim and payment data, and Pharmacy prescription data; Development of features for better preventative care and health management; and Growth of the MediKarma platform to cater to a broader audience and address various healthcare needs.

By combining these aspects, Jill and the MediKarma platform aim to revolutionize patient-facing healthcare, empowering individuals to take control of their health and well-being. Through the continuous development and improvement of the chatbot's capabilities and the platform's features, Jill will become an essential tool in personalized healthcare management and preventative care.

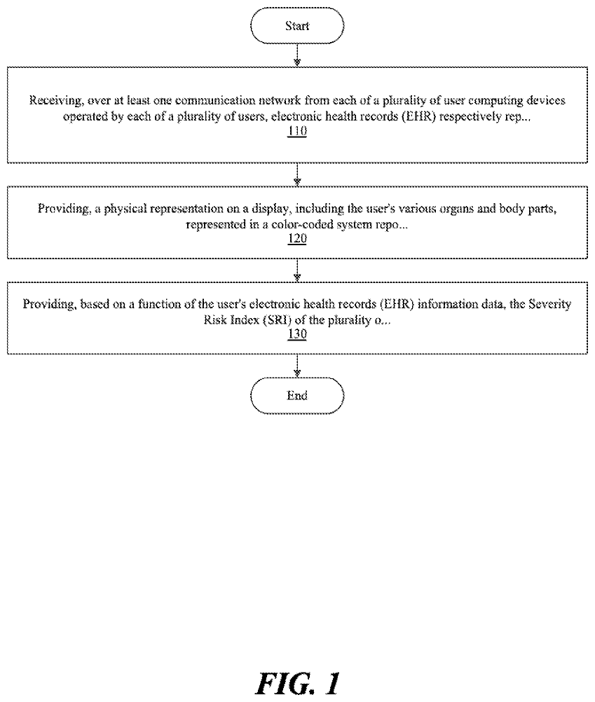

FIG. 1 is a flowchart that describes method of communicating, according to some embodiments of the present disclosure. In some embodiments, at 110, the method may include receiving a random query from a patient on any personal health-related concern. At 120, the method may include accessing a decentralized network for retrieving data specific to each of a plurality of users, comprising electronic health records (EHR) respectively representing information from the plurality of user's electronic medical records, procured from the user's health care provider or the user's electronic health records (EHR) partners.

In some embodiments, at 130, the method may include accessing a decentralized network to retrieve data from a plurality of smart wearable medical or sport devices. At 140, the method may include accessing a decentralized network to retrieve proprietary questionnaire data. At 150, the method may include accessing historical data of all past prior patient queries in a chat window. At 160, the method may include responding to the query from the patient by communicating critical health data retrieved from the decentralized network, assessing the patient's health, and providing next steps for the patient based on the patient's health assessment. The questionnaire data may include a daily health check survey. The historical data may be aggregated to train the AI to contextualize each new query.

In some embodiments, the artificial intelligence (AI) may be a human-like character with an avatar representing a health care expert named “Jill”. In some embodiments, the the avatar can be any character selected by a user, including any variation of customized features, selected from any one of race, gender, color, or creed. In some embodiments, the the avatar may use augmented reality technology to create an animation for the presentation of the response to the patient query.

In some embodiments, the response to the patient may include a plurality of ailments and conditions and further providing an explanation and/or prognosis of steps the patient may be recommended to take to address the plurality of ailments and conditions. In some embodiments, the artificial intelligence and natural language processing database may be web 3.0 compliant. In some embodiments, the avatar may provide pre-operation or post-operation instructions to the patient upon receiving the patient query.

In some embodiments, the decentralized network may comprise a library of frequently asked questions (FAQ) for all known health standards to date and may be trained according to standards stated in any one of the National Library of Medicine, Mayo Clinic, World Health Organization (WHO), WebMD, MedicinePlus or any other reputable authorities in medical training. In some embodiments, the artificial intelligence and natural language processing database may be equipped to identify and perform prescription refills utilizing prescription data.

In some embodiments, the prescription refill data may include which pharmacy chains and that may be available to fill the prescription. In some embodiments, the prescription refill data may include the location of the pharmacy available to fill the prescription. In some embodiments, the avatar identifies and provides preventive care through knowledge of the patient's chronic conditions and proactive/preventive tests and diagnostic tests and/or required vaccinations.

In some embodiments, the artificial intelligence avatar interacts with a patient avatar to present a representation of all of the ailments and conditions relevant to the patient. In some embodiments, electronic health records (EHR) respectively representing information from the plurality of user's electronic medical records, procured from the user's health care provider or the user's electronic health records (EHR) partners, generate a health score to alert the patient of patient's overall health assessment.

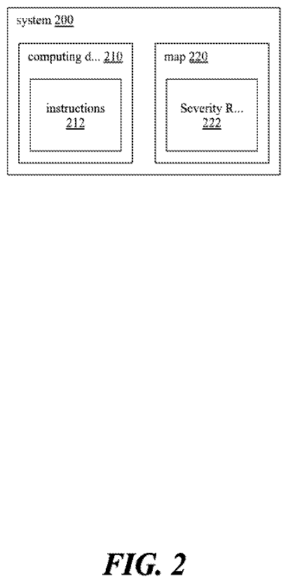

FIG. 2 is a block diagram that describes a system 210, according to some embodiments of the present disclosure. In some embodiments, the system 210 may include a computing device 212 configured to access non-transitory processor-readable media. The computing device 212 may also include instructions 214 that, when executed by the computing device 212 cause the computing device 212 to: The instructions 214 may also include electronic health records 216 (EHR) respectively representing information from the plurality of user's electronic medical records, procured from the user's health care provider or the user's electronic health records (EHR) partners.

In some embodiments, a decentralized and distributed method of communicating and providing robust critical formulation of health-related clinical data of a patient using artificial intelligence (AI) and natural language processing (NLP) is used to engage a patient. After, Jill receives a random query from a patient on any personal health-related concern, she then begins accessing a decentralized network for retrieving data specific to each of a plurality of users to determine and overall health assessment.

In some embodiments, the processor accesses a decentralized network to retrieve data from a plurality of smart wearable medical or sport devices. After Jill obtains access to the decentralized network to retrieve proprietary questionnaire data 220, Jill will also access historical data of all past prior patient queries in a chat window to obtain context for the WDIDN instructions. The historical data may be aggregated to train the AI to contextualize each new query. And. Responding to the query from the patient by communicating critical health data retrieved from the decentralized network, assessing the patient's health, and providing next steps for the patient based on the patient's health assessment. The system 210 of claim 15, the artificial intelligence (AI) may be a human-like character with an avatar representing a health care expert character named “Jill”. The questionnaire data 220 may include a daily health check survey 222.

In some embodiments, the avatar may use augmented reality technology to create an animation for the presentation of the response to the patient query. In some embodiments, the artificial intelligence and natural language processing database may be web 3.0 compliant. In some embodiments, the avatar may provide pre-operation or post-operation instructions to the patient upon receiving the patient query.

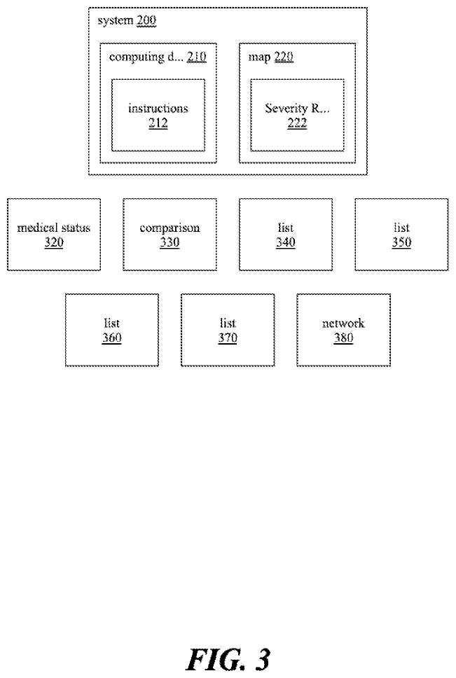

FIG. 3 is a block diagram that further describes the system 210 from FIG. 2, according to some embodiments of the present disclosure. In some embodiments, the response to the patient may be spoken and/or displayed on an application of a mobile device or desktop.

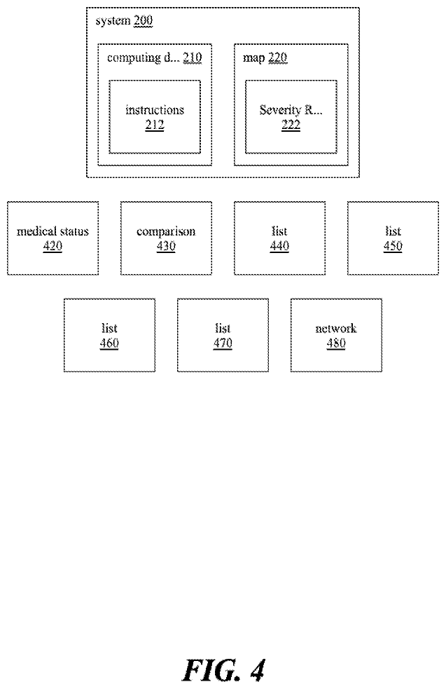

FIG. 4 demonstrate how system 2 will be used to generate a personalized recommendation (also called “we do I do next” or “WDIDN” for short) for the user. The recommendation interface will inform of the specific actions the user should take to improve their heath and associated health score. The WDIDN interface also shows the user their health score trend over the last year as well as the impact of the specific health condition on their health score.

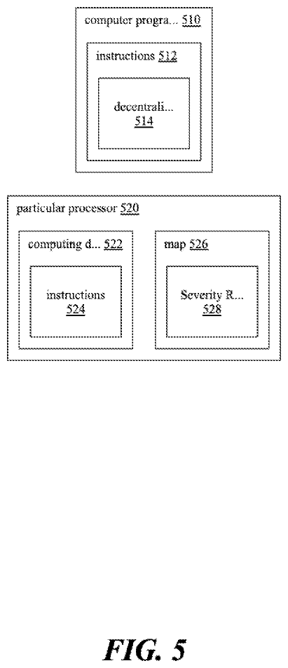

FIG. 5 demonstrates a health score history of the patients progresses on a monthly basis. The next section is the WDIDN section which describes the progress of the steps that the patient has taken and the impact on their score. The WDIDN section gives specific information using the electronic medical records (EHR) on the exact ailment that is of the highest priority and gives background information of the ailment and possible factors to consider. The WDIDN section also shows the patient other categories that the patient may have improved on that may increase the patient's overall health score. The information is presented to the patient through Jill. Jill is an artificial intelligence advance system that provides the information in the form of a conversation with the patient. This human feel helps the patient to connect and relate with the information being presented by Jill.

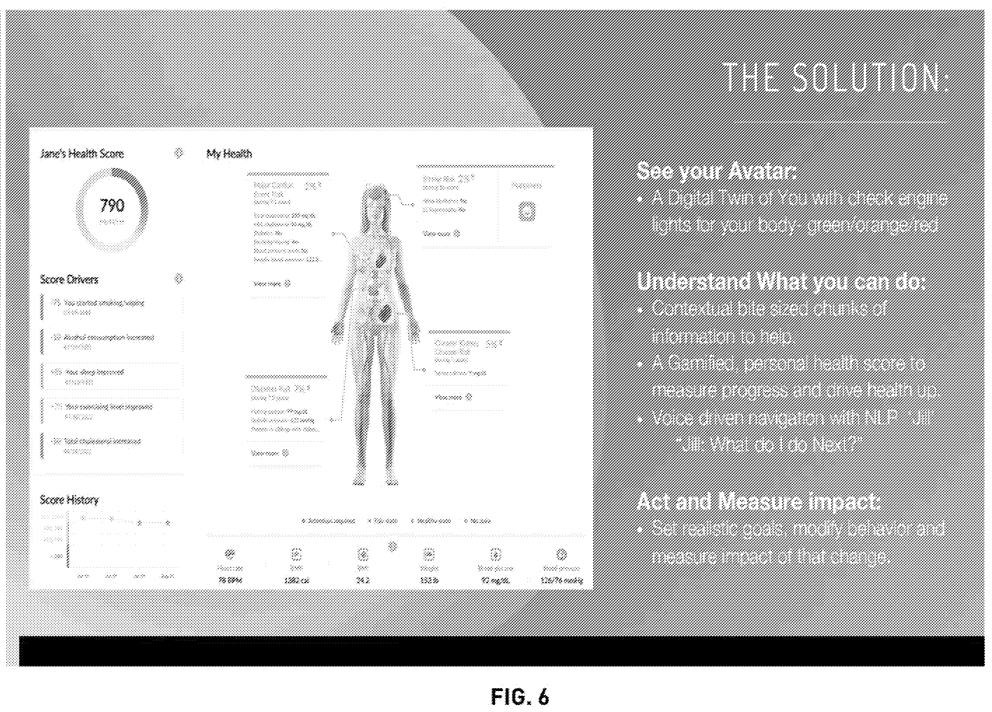

FIG. 6 demonstrates how the avatar of the Jill is the ultimate virtual health assistant from MediKarma. Jill is at the fingertips of the patient and provides all the information in existence related to the patient's health. The natural language processing allows for the seamless engagement with the patient and the patients'health related data. The AI is always learning new information about the patient and determining ways to implement insights through an overall score. The data is complimented with graphs and charts that are analogous to a check engine light in a car's dashboard. The data also includes personalized recommendations for behavioral changes that could help to improve the patient's overall health.

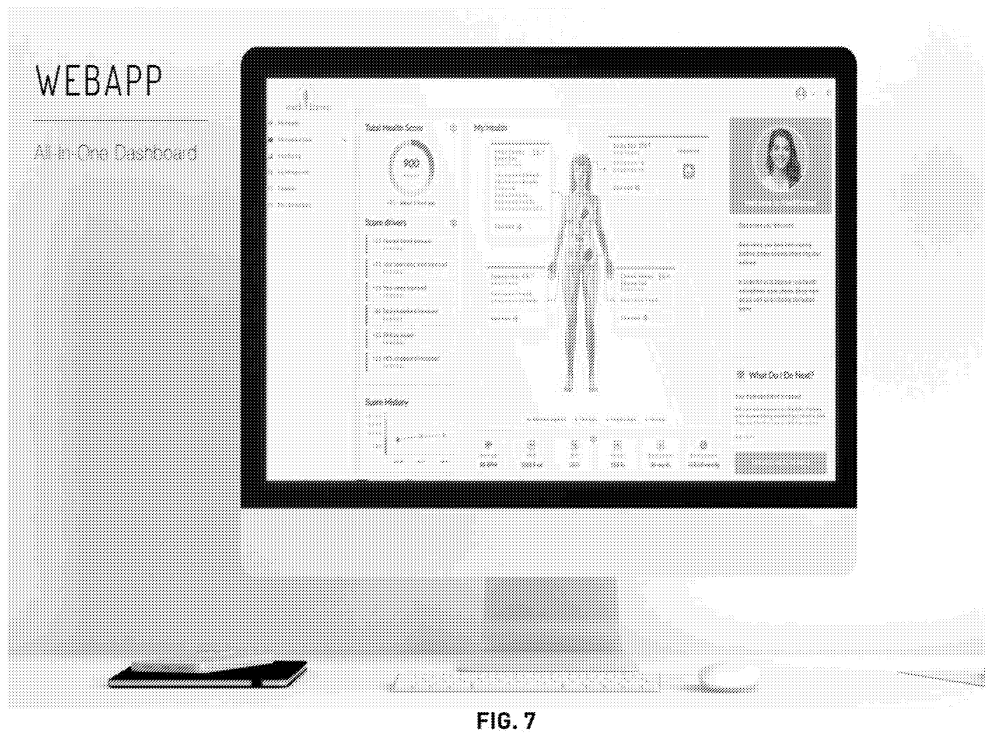

FIG. 7 demonstrates how MediKarma is a generative-AI platform that delivers health information in natural language, empowering people to take preventative care of their health. FIG. 7 displays a conversation between Jill and the patient. Jill is positive and accurate about the future health of the patient after following recommendations that relate to the patient.

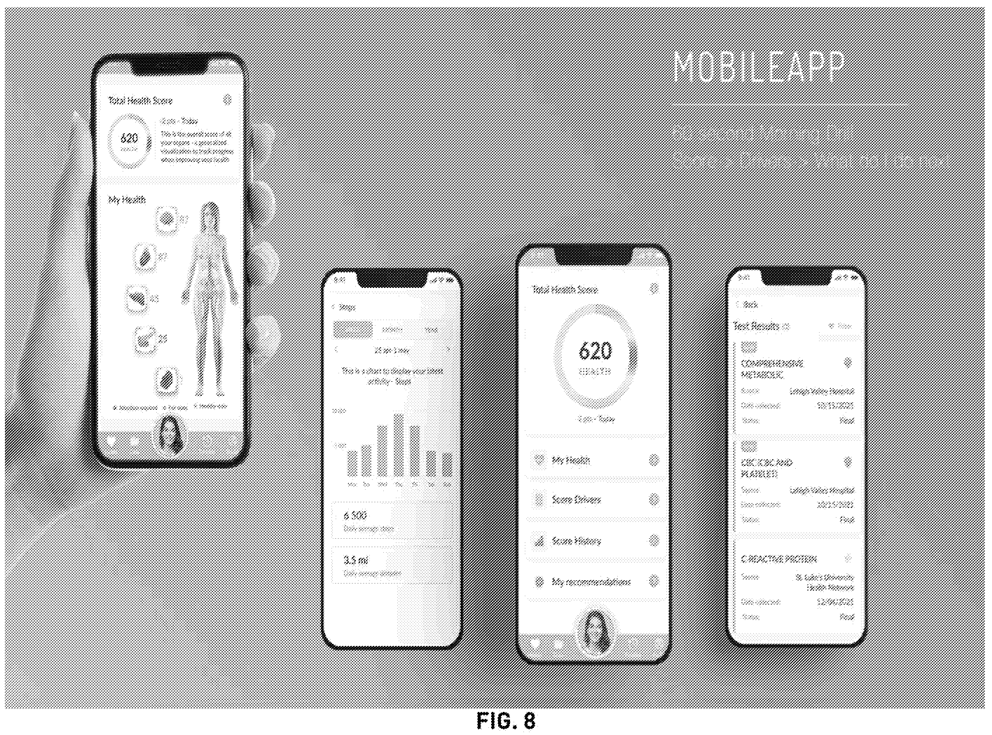

FIG. 8 demonstrates how advance Jill is in comprehending health data, deciphering technical terms and extracting meaning insights. With access to all available health data related to the patient, the patient is empowered to take control of their own health. Data is collected from records, wearables, and other devices. Jill acts as a health coach by consistently providing tailored assessments of the patient's health. Jill is a health concierge that handles tasks like scheduling appointments at pharmacies, or a telehealth visits using simple voice commands spoken to the application.

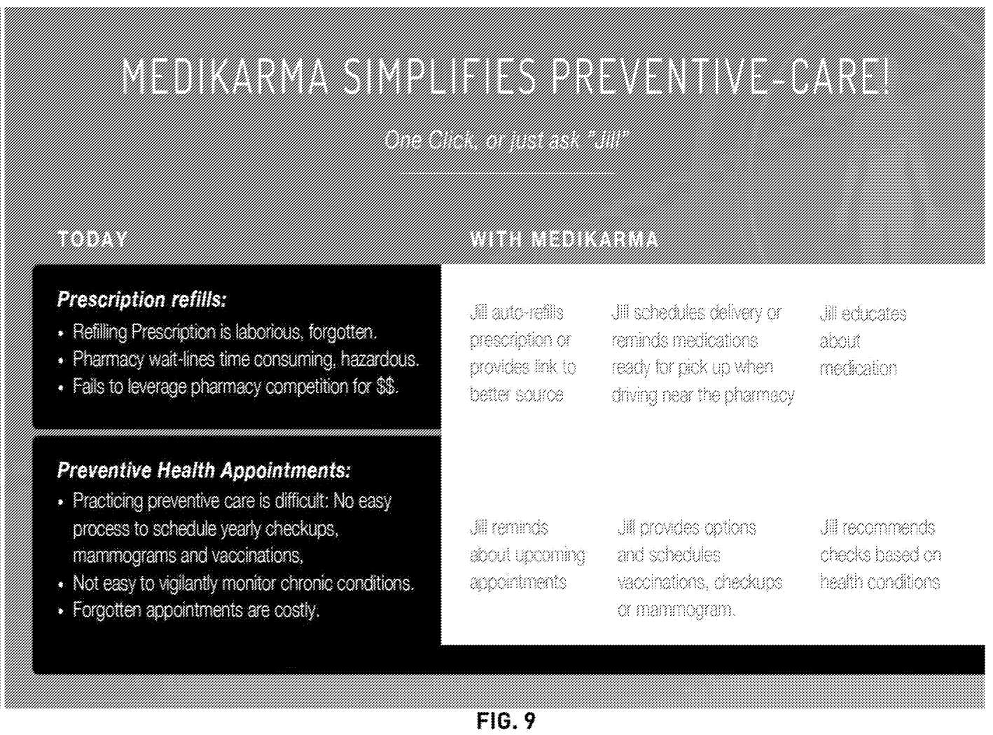

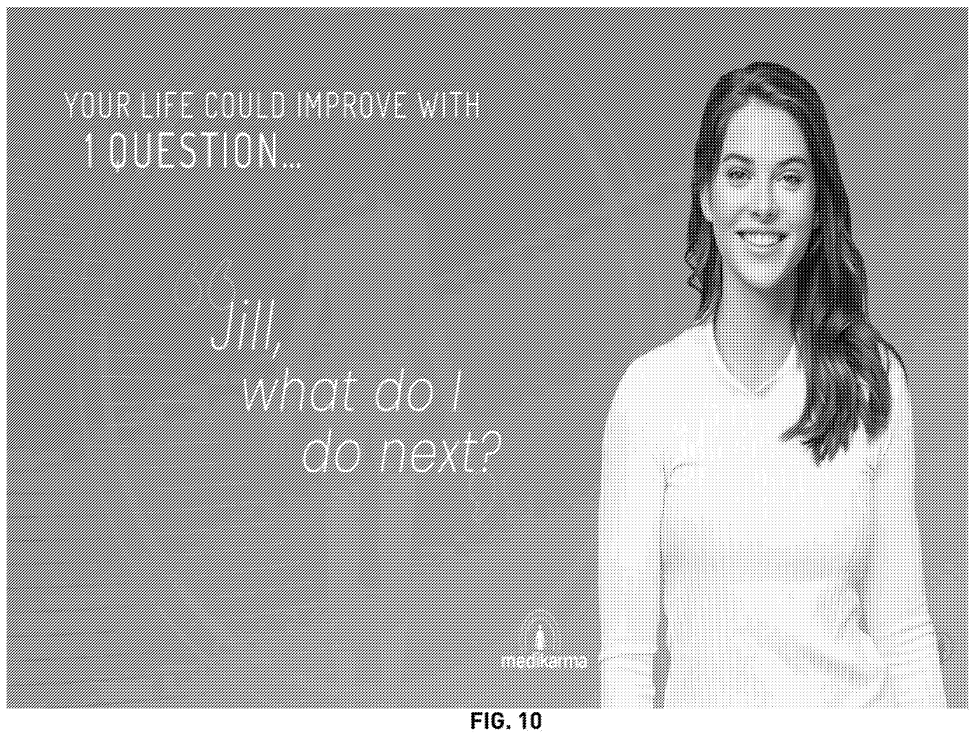

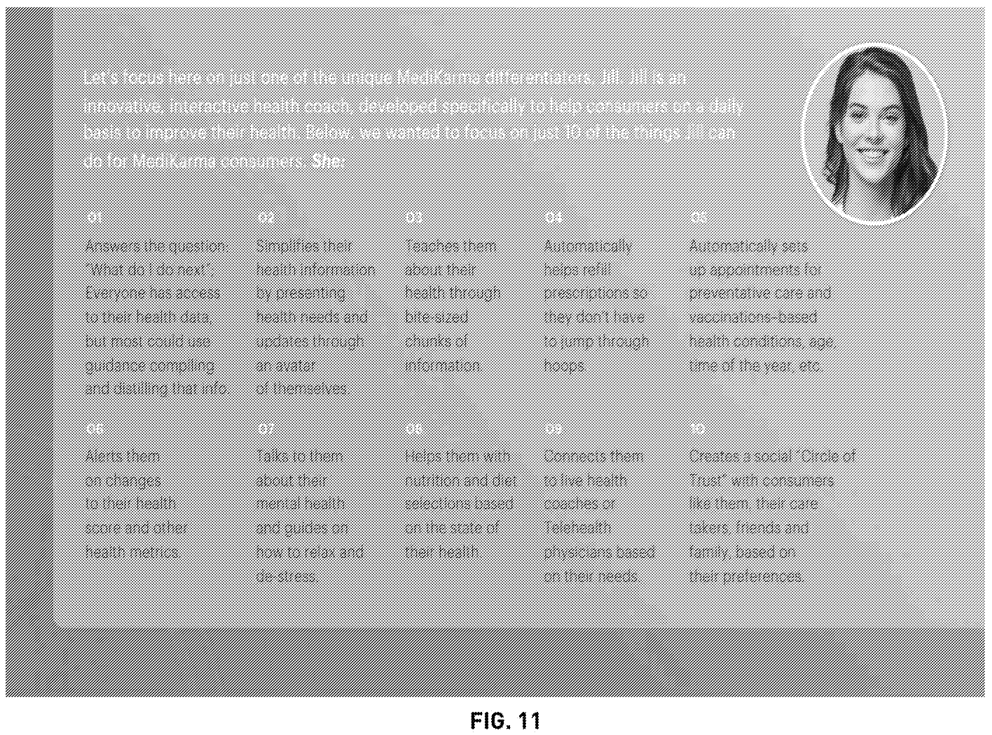

FIG. 9 demonstrates a visual representation of the interface the patient will use to present the visualization of Jill and how it would interact with the patient on a mobile application. The figure depicts various pages on the MediKarma application, which includes a health score and a visual representation of the patient's body with each issue the patient is experiencing at the time of assessment.

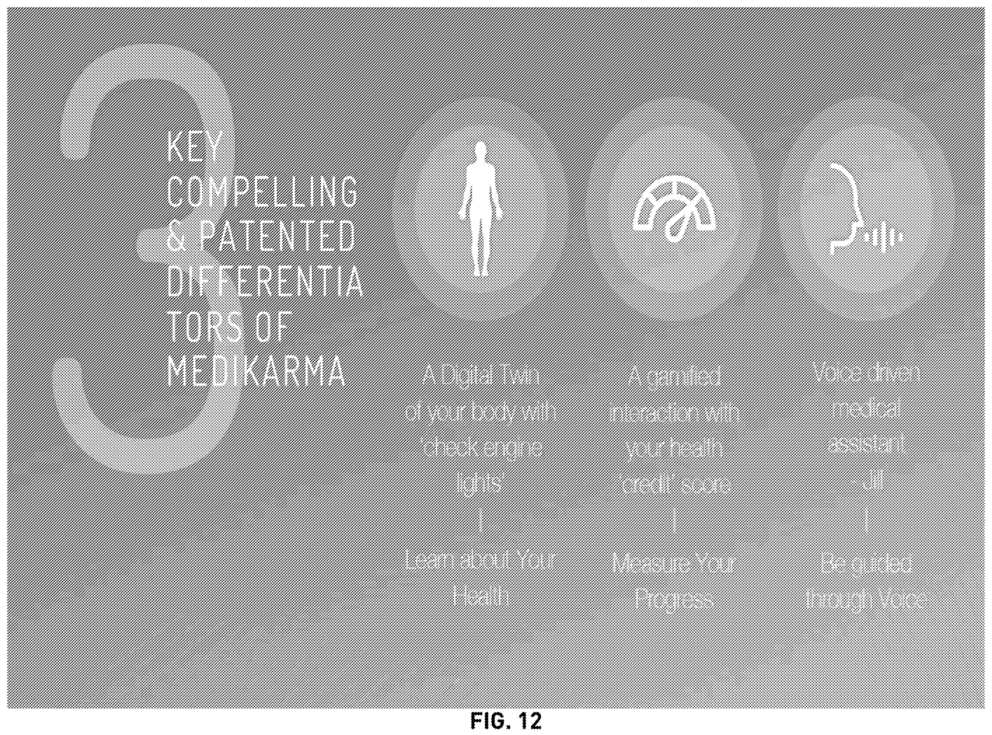

FIG. 10 demonstrates a visual representation of the interface the patient will use to present the visualization of Jill and how it would interact with the patient on a desktop. The figure depicts various pages on the MediKarma desktop application, which includes a health score and a visual representation of the patient's body with each issue the patient is experiencing at the time of assessment.

What is claimed is:

1. A decentralized and distributed patient facing method of communicating and providing robust critical formulation of health-related clinical data of a patient using artificial intelligence (AI) and natural language processing (NLP) to engage a patient, comprising:
receiving a random query from a patient on any personal health-related concern;
accessing a decentralized network for retrieving data specific to each of a plurality of users, comprising electronic health records (EHR) respectively representing information from the plurality of user's electronic medical records, procured from the user's health care provider or the user's electronic health records (EHR) partners;
accessing a decentralized network to retrieve data from a plurality of smart wearable medical or sport devices;
accessing a decentralized network to retrieve proprietary questionnaire data, wherein the said questionnaire data includes a daily health check survey;
accessing historical data of all prior patient queries in a chat window, wherein the historical data is aggregated to train the AI to contextualize each new query;
and
responding to the query from said patient by communicating critical health data retrieved from the decentralized network, assessing the patient's health, and providing next steps for the patient based on the patient's health assessment.

2. The decentralized and distributed method of claim 1, said artificial intelligence (AI) is a human-like character with an avatar representing a health care expert named “Jill”.

3. The decentralized and distributed method of claim 2, wherein the said avatar can be any character selected by a user, including any variation of customized features, selected from any one of race, gender, color, or creed.

4. The decentralized and distributed method of claim 2, wherein the said avatar uses augmented reality technology to create an animation for the presentation of said response to said patient query.

5. The decentralized and distributed method of claim 1, wherein the query response to said patient may include a plurality of ailments and conditions and further providing an explanation and/or prognosis of steps said patient is recommended to take to address the plurality of ailments and conditions.

6. The decentralized and distributed method of claim 1, wherein the artificial intelligence and natural language processing database are web 3.0 compliant.

7. The decentralized and distributed method of claim 1, wherein said avatar provides pre-operation or post-operation instructions to said patient upon receiving said patient query.

8. The decentralized and distributed method of claim 1, wherein said decentralized network comprises a library of frequently asked questions (FAQ) for all known health standards to date and is trained according to standards stated in any one of the National Library of Medicine, Mayo Clinic, World Health Organization (WHO), WebMD, MedicinePlus or any other reputable authorities in medical training.

9. The decentralized and distributed method of claim 1, wherein the artificial intelligence and natural language processing database are equipped to identify and perform prescription refills utilizing prescription data.

10. The decentralized and distributed method of claim 9, wherein said prescription refill data includes which pharmacy chains and that are available to fill the prescription.

11. The decentralized and distributed method of claim 10, wherein said prescription refill data includes the location of the pharmacy available to fill the prescription.

12. The decentralized of claim 1, wherein the avatar identifies and provides preventive care through knowledge of said patient's chronic conditions and proactive/preventive tests and diagnostic tests and/or required vaccinations.

13. The decentralized of claim 1, wherein said artificial intelligence avatar interacts with a patient avatar to present a representation of all of the ailments and conditions relevant to the patient.

14. The decentralized of claim 1, wherein electronic health records (EHR) respectively representing information from the plurality of user's electronic medical records, procured from the user's health care provider or the user's electronic health records (EHR) partners, generate a health score to alert said patient of patient's overall health assessment.

15. A system comprising a computing device configured to access non-transitory processor-readable media having instructions that, when executed by the computing device cause the computing device to: provide a decentralized and distributed patient facing method of communicating and providing robust critical formulation of health-related clinical data of a patient using artificial intelligence (AI) and natural language processing (NLP) to engage a patient, comprising:
receiving a random query from a patient on any personal health-related concern;
accessing a decentralized network for retrieving data specific to each of a plurality of users, comprising electronic health records (EHR) respectively representing information from the plurality of user's electronic medical records, procured from the user's health care provider or the user's electronic health records (EHR) partners;
accessing a decentralized network to retrieve data from a plurality of smart wearable medical or sport devices;
accessing a decentralized network to retrieve proprietary questionnaire data, wherein the said questionnaire data includes a daily health check survey;
accessing historical data of all past prior patient queries in a chat window, wherein the historical data is aggregated to train the AI to contextualize each new query;
and
responding to the query from said patient by communicating critical health data retrieved from the decentralized network, assessing the patient's health, and providing next steps for the patient based on the patient's health assessment.

16. The system of claim 15, wherein the artificial intelligence (AI) is a human-like character with an avatar representing a health care expert character named “Jill”.

17. The system of claim 16, wherein the said avatar can be any character selected by a user, including any variation of customized features, selected from any one of race, gender, color, or creed.

18. The system of claim 16, wherein the said avatar uses augmented reality technology to create an animation for the presentation of said response to said patient query.

19. The system of claim 15, wherein the query response to said patient may include a plurality of ailments and conditions and further provide an explanation and/or prognosis of steps said patient is recommended to take to address the plurality of ailments and conditions.

20. The system of claim 15, wherein the artificial intelligence and natural language processing database are web 3.0 compliant.

21. The system of claim 15, wherein said avatar provides pre-operation or post-operation instructions to said patient upon receiving said patient query.

22. The system of claim 15, wherein said decentralized network comprises a library of frequently asked questions (FAQ) for all known health standards to date and is trained according to standards stated in any one of the National Library of Medicine, Mayo Clinic, World Health Organization (WHO), WebMD, MedicinePlus or any other reputable authorities in medical training.

23. The system of claim 15, wherein the artificial intelligence and natural language processing database are equipped to identify and perform prescription refills utilizing prescription data.

24. The system of claim 15, wherein said prescription refill data includes which pharmacy chains and that are available to fill the prescription.

25. The system of claim 15, wherein said prescription refill data includes the location of the pharmacy available to fill the prescription.

26. The system of claim 15, wherein the avatar identifies and provides preventive care through knowledge of said patient's chronic conditions, proactive/preventive tests and diagnostic tests, and/or required vaccinations.

27. The system of claim 15, wherein said artificial intelligence avatar interacts with a patient avatar to present a representation of all of the ailments and conditions relevant to the patient.

28. The system of claim 15, wherein electronic health records (EHR) respectively representing information from the plurality of user's electronic medical records, procured from the user's health care provider or the user's electronic health records (EHR) partners, generate a health score to alert said patient of patient's overall health assessment.
